(19) United States
(12) Patent Application Publication
(10) Pub. No.: US 2015/0090063 A1
(43) Pub. Date: Apr. 2, 2015

(54) ROBOTIC ARRANGEMENT FOR USE IN MEDICAL FIELDS

(71) Applicant: Deutsches Zentrum Fur Luft-und Raumfahrt E.V., Koeln (DE)

(72) Inventors: Sophie Lantermann, Muenchen (DE); Ulrich Hagn, Muenchen (DE); Ulrich Seibold, Burnaby (CA); Georg Passig, Kosching (DE); Florian Froehlich, Germering (DE)

(21) Appl. No.: 14/396,431

(22) PCT Filed: Apr. 22, 2013
(86) PCT No.: PCT/EP2013/058282

§ 371 Date: Oct. 23, 2014

Related U.S. Application Data
(60) Provisional application No. 61/711,889, filed on Oct. 10, 2012.

(30) Foreign Application Priority Data
Apr. 27, 2012 (DE) 102012207060.5

(51) Int. Cl. B25J 19/00; A61B 19/00

(52) U.S. Cl. CPC B25J 19/0025; A61B 19/2203
USPC 74/490.02

(57) ABSTRACT

A robotic arrangement for use in medical fields has a robot arm having, in particular, a plurality of hinges, an instrument holder provided on the robot arm in order to receive a medical instrument, and supply lines which can be connected to the instrument. An adapter element is used for connecting the supply lines to the instrument.

### BACKGROUND

1. Field of the Disclosure

The disclosure relates to a robotic arrangement for use in medical fields. In particular, the robotic arrangement is suited for use in sterile areas, such as operating rooms.

2. Discussion of the Background Art

In surgery, instruments that come into contact with a patient or the interior of a patient have to be sterile, i.e. free from living microorganisms. Various methods exist for the sterilization of devices. For example, such methods include vapor sterilization, hot air sterilization, fractionated sterilization, chemical sterilization, radiation sterilization or plasma sterilization. The sterilization most often takes place outside the operating area so that the devices are moved into the same in a sterile condition. However, some devices cannot be sterilized because of their structure or size. For example, medical robots, frequently used in surgery today, include electronic components that could be damaged by conventional sterilization methods.

In order to avoid that non-sterilized surfaces of medical robots contaminate sterilized devices or instruments, medical robots are usually packed in a sterile condition in situ.

Most often, sterile packaging materials such as films are used for the sterile packages for devices, such as medical robots, these materials also being referred to as drapes. Generally speaking, a drape is a sterilized film hose pulled over the technical device and fixed thereon. In this regard it is of particular importance that the drape is not damaged while it is applied and that the sterile surface of the drape facing the sterile side of the operating area is not contaminated.

Further, a surgical manipulator for robotic systems is known from DE 696 35 050. Here, an instrument holder is provided between the instrument and the robot arm to releasably hold an instrument. Thus, force is transmitted from the robot arm or the individual elements of the robot arm to the instrument holder and from there to the instrument.

There is a general problem that the supply lines, such as electric wires, data transmission lines and hoses for medium supply and discharge, have to be connected to the instrument. The supply lines enable the supply or discharge of media, for example, such as liquids and gases. Further, the supply lines may also include light conductors, laser conductors, HF, plasma etc. In this regard, the corresponding supply lines are either guided outside the robot arm or inside the robot arm. Guiding the lines inside the robot arm is disadvantageous in that the available space is limited. Therefore, only a limited number of supply lines can be integrated into the robot arm. In case of the supply lines being guided outside the robot arm, the supply lines can be connected permanently to the instrument or may be detachable from the same. This separation may be useful, since the supply lines can thus be used again, remain permanently connected to the medical instruments and fewer lines are present in the operating area. For this purpose, the connection between the supply lines and the instrument must be releasable, so that another instrument can be connected to the supply lines. While the supply lines are detached from or reconnected to the instruments, sterility problems can occur.

It is an object of the present disclosure to provide a robotic arrangement for use in medical fields, which enables a simple change of instruments, while observing the necessary sterility requirements and allowing the reuse of the supply lines, if so desired.

### SUMMARY

The robotic assembly for use in medical fields, in particular in sterile areas such as operating rooms, for example, comprises a robot arm having a plurality of hinges, for example. An instrument holder is arranged on the robot arm to receive a medical instrument. According to the disclosure, the supply lines connectable to the instrument are not connected directly to the instrument, but are connected thereto via an adapter element. Possibly, according to the disclosure, a part of the supply lines is connected directly to the instrument, while another part of the supply lines is connected indirectly to the instrument via the adapter element. Preferably, all supply lines are connected to the instrument via the adapter element. This has the advantage provided by the disclosure that in order to change an instrument, the same merely has to be detached from the adapter element and the new instrument has to be set on or connected to the adapter element. By providing the adapter element, the disclosure remedies the disadvantage of a separate connection, in particular of a plurality of supply lines, plug-in connectors or the like to the instrument, and the sterility and manipulation disadvantages associated therewith.

The transmission of force onto the instrument, such as the transmission of forces and moments for moving the instrument by means of the robot arm, can be effected directly from the robot arm to the instrument holder or from the robot arm to the instrument holder via the adapter element. In a particularly preferred embodiment, the forces and moments are transmitted directly from the robot arm to the instrument. Thus, in a particularly preferred embodiment, the adapter element is at least substantially free from force influences.

In order to enable a change of instruments that is as simple as possible, it is particularly preferred that all instrument supply lines are connected to the adapter element.

The adapter element can be connected to the robot arm together with the instrument via a common holder element on the instrument holder. In this regard, it is possible to provide a connection such as holding the instrument and the adapter element by means of magnets, for example, wherein the magnets are arranged such that no forces or only negligible forces act on the adapter element. For a further reduction of an unintentional transmission of force onto the adapter element and, in particular, to exclude the same entirely, it is particularly preferred that the adapter element and the instrument are separately connected with the instrument holder via holder elements, in particular mechanical holder elements.

The connection of the supply lines to the instrument, which is made via the adapter element, can be effected such that when the instrument is connected to the instrument holder, the instrument is at the same time connected to the adapter element and thereby the supply lines are connected to the instrument. Thus, when the instrument is coupled to the instrument holder, a connection to the supply lines is effected at the same time, e.g. by means of a plug connection. However, a spatial/temporal separation of the connecting operations can be provided, so that the instrument can be independently connected to the instrument holder and to the adapter element. Thereby, a further decoupling of forces can possibly be ensured. Even if in this embodiment an additional connecting operation, such as plugging the supply lines, is required when the instruments are changed, this is still simpler than in prior art, since spatial proximity is ensured. In particular, a plurality of supply lines or ports of the instrument can be connected to the adapter element at the same time, and can thus be connected to a plurality of supply lines at the same time. Further, it is avoided that the supply lines become caught, as could happen if a plurality of instruments is used and is changed. Moreover, it is not necessary the supply lines to plug the supply lines into the devices again and the number of ports in the devices is reduced.

In a preferred embodiment of the disclosure, the adapter element can be of a modular structure. In this case, the adapter element comprises a plurality of adapter modules, each connected to at least one supply line. By assembling the adapter modules in a corresponding manner, an adapter element adapted to an individual instrument, a set of instruments or a specific operation can be put together in a simple manner. This can be done, for example, while preparing an operation, since the instruments used are known from the surgery plan.

Preferably, the adapter element can comprise a standard adapter element that can be connected with one ore a plurality of additional adapter elements. With such a modular structure, the otherwise always necessary ports, such as a connection to a data transmission line etc., can be realized via the standard adapter element. The additional adapter elements of different design can then be added depending on the current requirements. In this regard, the standard adapter element can be designed such that it is at least partially surrounded by the additional adapter element. The additional adapter element can be in the form of a ring or a partial ring, the additional adapter element preferably being rotatable about the standard adapter element. Due to this in particular free rotatability of the additional adapter element about the standard adapter element, no movement of the supply lines is required, preferably when the instrument is turned. In this regard, the connection of the supply lines can be realized by annular ducts or the like. Generally, power transmission can be effected via sliding contacts, plug-in contacts, spring contacts, in an inductive or optical manner using coils. In this context, the corresponding power transmission can be effected in particular between the adapter element or individual modules of the adapter element and the instrument.

It is further possible to design the adapter element in such a modular manner that individual adapter modules are arranged in the axial direction, i.e. as a series of disc-shaped elements.

Owing to the presence of an adapter element, as provided by the disclosure, a good sterilizability of the instruments can be achieved. Further, the robot arm and the supply lines connected with the adapter element can be enclosed by separate sterile drapes, i.e. sterilized hose elements. The supply lines and the robot arm can also have a common drape, with the provision of separate or double-lumen drapes being preferred, since in this case, for example, liquids leaking from supply lines are prevented from entering the hinges of the robot arm. The individual supply lines can be packed in a sterile condition in separate drapes. However, it is also possible to sterilely pack a plurality of groups of supply lines in a common drape, in particular a plurality of supply line groups connected with an adapter module.

It is further preferred that the surgical instrument is driven and is therefore connected with corresponding lines, in particular for communication.

In a particularly preferred embodiment of the robotic arrangement of the disclosure, the medical instrument is driven. Thus, for the purpose of being driven, the instrument is connected with power supply lines and/or communication lines for data communication. In this particularly preferred embodiment, the instrument can be connected with very different supply lines via the adapter element.

It is particularly advantageous that the robotic arrangement of the disclosure enables the realization of a variable coupling of supply lines to the surgical instrument. In particular, the adapter element can be detached from the drive unit so that various embodiments can be coupled thereto. Further, the adapter element can be designed such that different supply modules can be installed, thereby making the adapter element adaptable to the requirements of a surgical operation.

The following is a detailed description of the disclosure with reference to preferred embodiments thereof and to the accompanying drawings.

### BRIEF DESCRIPTION OF THE DRAWINGS

In the Figures:

FIG. 1 schematically illustrates a preferred embodiment of a robotic arrangement,

FIGS. 2 and 3 show different possible ways of fastening the instrument and the adapter element to the instrument holder, and

FIGS. 4 to 8 show different preferred embodiments of the adapter element.

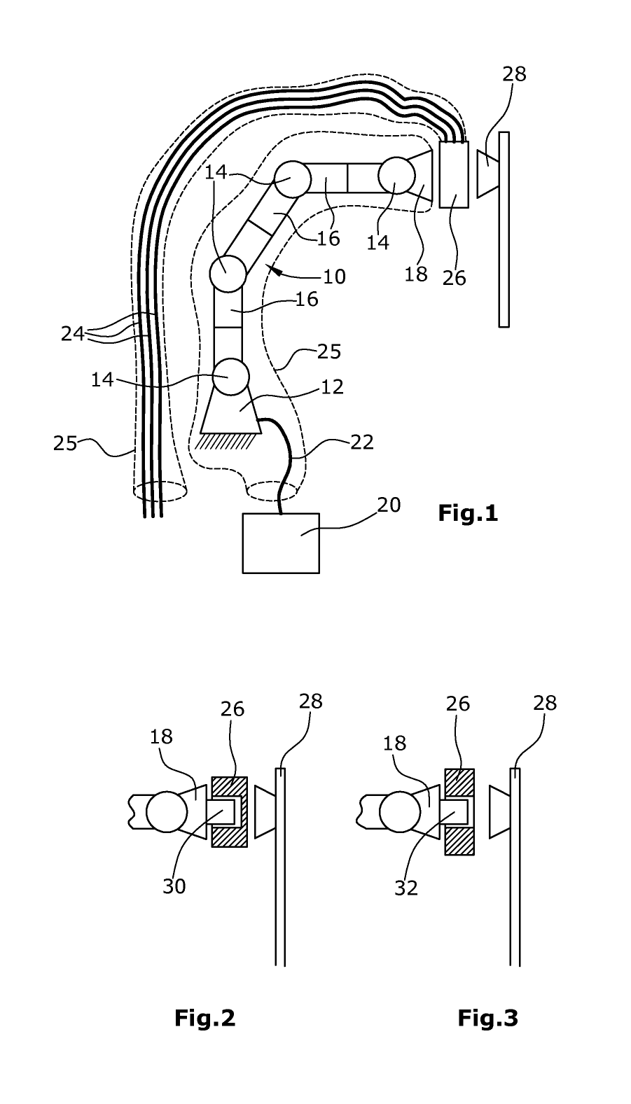

### DETAILED DESCRIPTION OF THE PREFERRED EMBODIMENT

A robotic arrangement comprises a robot arm 10 having, in the embodiment illustrated, a stationary base element 12 and a plurality of arm links 16 connected by hinges 14. An instrument holder 18 is arranged on the free end of the robot arm 10. The robot arm 10 is connected with a control means 20 via an electric cable 22. The cable 22 extends inside the robot arm 10 and supplies electric power in particular to the individual actuators and the electronics. The robot arm 10 is surrounded by a drape 22, i.e. a sterile cover.

Different supply lines 24 are guided to an adapter element 26. The supply lines 24 may be electric wires, optical conductors, data transmission cables and hoses for the supply and the discharge of medium. In the embodiment illustrated, all supply lines 24 are jointly surrounded by one drape 25.

It is likewise possible to provide a common drape for the supply lines 24 and the robot arm 10 or a double-lumen drape. Similarly, the lines 24 can each be individually surrounded by a drape.

The instrument 28 can be controlled via the control means 20 illustrated or via a separate control means not illustrated.

Both the adapter element 26 and an instrument 28 are connected to the instrument holder. The connection between the instrument holder 18, the adapter element 26 and the instrument 28 can be made at the same time. For the purpose of a simultaneous fixation, a magnet 30 can be provided, by which both the instrument 28 and the adapter element 26 are fixed to the instrument holder 18. Thus, a connection with the corresponding ports of the adapter element can be made during the very fixation of the instrument 28, so that a connection between the supply lines 24 and the instrument 28 is directly established.

It is further possible to design the adapter element 26 in an annular shape (FIG. 3), wherein a separate, in particular mechanical connection is made between the instrument holder 18 and the adapter element 26 and between the instrument holder 18 and the instrument 28.

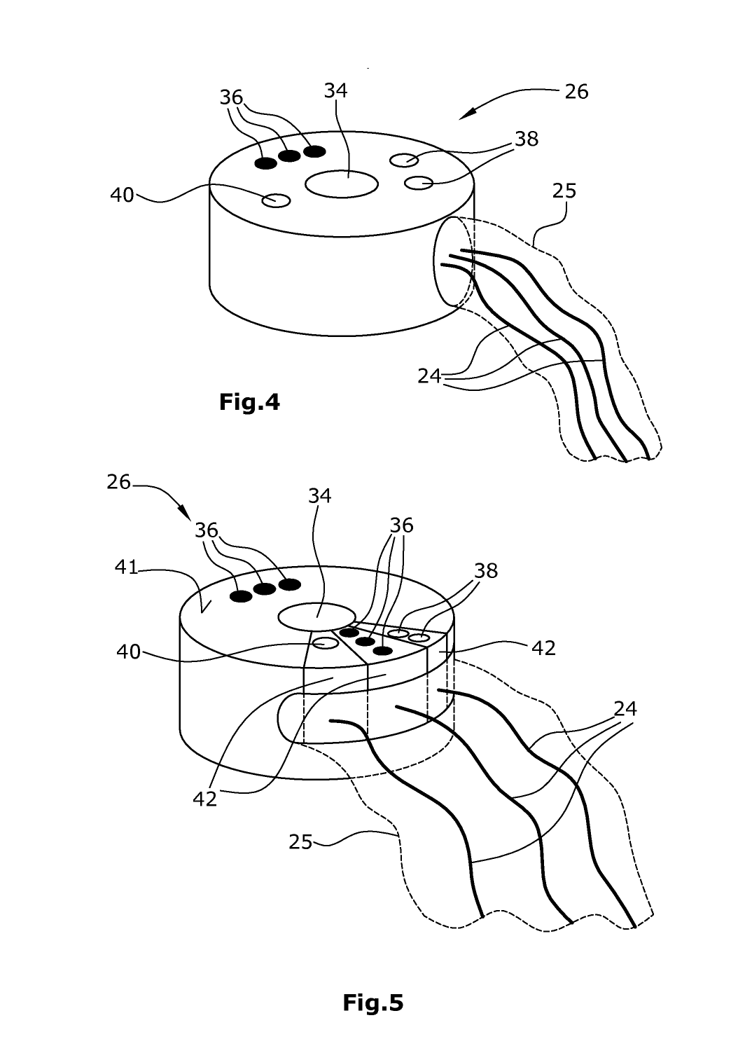

An adapter element 26 is preferably connected with a plurality of supply lines 24 sheathed in a drape 25. The adapter element can be annular in shape (FIG. 4) so that a protrusion 32 (FIG. 3) of the instrument holder 18 extends through a cylindrical central opening 34.

Thus, a part of the protrusion 32 serves for connection with the instrument 28. The adapter element 26 annularly surrounds the protrusion 32. The adapter element has different connection points 36, 38, 40. These are plug-in connections, for example. Depending on the type of instrument 28 used, these are connected in particular immediately upon fixation of the instrument.

Another embodiment of the adapter element (FIG. 5) is also annular in shape and has a corresponding opening 34. However, the adapter element illustrated in FIG. 5 is of modular design. Individual adapter modules 42 having a ring segment-shaped cross section, for example, can be exchanged in a simple manner so that different adapter elements 26 can be assembled that are easily adaptable to the corresponding requirements. The individual adapter modules 42 also have different plug-in connections 36, 38, 40. In this regard, the supply lines to the adapter modules can be packed in one or a plurality of drapes.

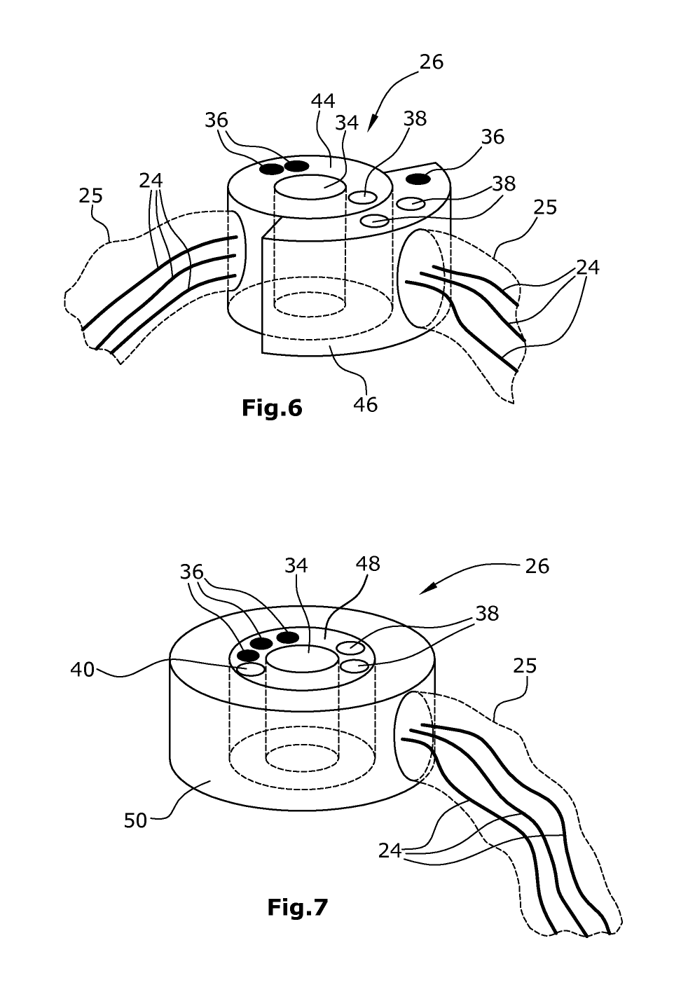

Further, a modular structure of the adapter element 26 is also possible in such a manner that a standard module 44 (FIG. 6) is provided which is illustrated in an annular shape in the embodiment illustrated. The same is connected with corresponding supply lines 24 and is provided with plug-in connections 36. An additional adapter module 46, which in the embodiment illustrated is of a ring segment shape, is also provided with supply lines 24 and, in the embodiment illustrated, has the plug-in connections 36, 38. It is also possible that a plurality of ring segment-shaped adapter modules 46 is arranged around an inner ring segment-shaped adapter module 44.

According to another embodiment (FIG. 7) of the adapter element 26, the same has an inner annular adapter module 48 comprising all plug-in connections 36, 38, 40. The inner annular adapter module 48 is surrounded by a further annulus that is connected with the supply lines 24. Preferably, the outer annulus 50 is rotatable with respect to the inner annular adapter module 34. In particular, the outer annulus 50 is freely rotatable. The connection between the supply lines and the plug-in connections 36, 38, 40 can be made by sliding rings, ring ducts or the like. Due to the free rotatability of the outer ring element 50, it is possible to rotate the instrument 28 in the ring 50 together with the adapter module 48, so that no movement of the supply lines 24 is required during rotation.

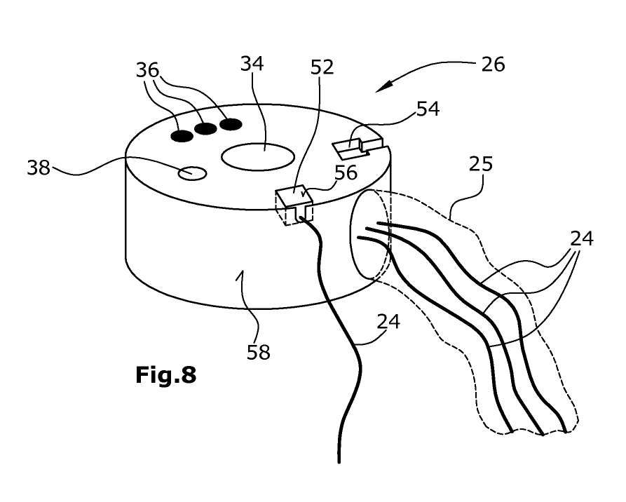

In a further preferred embodiment (FIG. 8), identical or similar components are identified by the same reference numerals. It is the particularity of this embodiment that adapter modules 52 are placed inside the adapter element 26. For this purpose, the adapter element 26 has correspondingly shaped recesses 54. In this regard, a supply line 24 connected with the adapter module 52 can be packed separately in a drape. A surface 56, which may not be sterilizable, does not come into contact with the tool or instrument that comes into contact with a patient. Only the outer surface 58 of the sterilizable adapter element 26 will come into contact with the instrument or the patient.

What is claimed is:

1. A robotic arrangement for use in medical fields, comprising:
a robot arm having a plurality of hinges,
an instrument holder provided on the robot arm in order to receive a medical instrument, and
supply lines which can be connected to the instrument, wherein
an adapter element for connecting the supply lines to the instrument.

2. The robotic arrangement of claim 1, wherein all instrument supply lines are connected to the adapter element.

3. The robotic arrangement of claim 1, wherein, for the purpose of force transmission, the instrument is connected substantially the instrument holder.

4. The robotic arrangement of claim 1, wherein the adapter element and the instrument are connected to the instrument holder via a common holder element.

5. The robotic arrangement of claim 1, wherein the adapter element and the instrument are connected separately to the instrument holder via mechanical holder elements.

6. The robotic arrangement of claim 1, wherein the adapter element comprises a plurality of adapter modules respectively connected to at least one supply line.

7. The robotic arrangement of claim 1, wherein the adapter element comprises a standard adapter element adapted to be connected to additional adapter elements.

8. The robotic arrangement of claim 7, wherein the additional adapter elements surround the standard adapter element annularly and are preferably rotatable around the standard adapter element.

9. The robotic arrangement of claim 1, wherein the robot arm and/or supply lines leading to the adapter element are surrounded by a sterile drape, possibly with multiple lumens, or by separate drapes.

10. The robotic arrangement of claim 1, wherein, for the purpose of driving the instrument, the medical instrument is connected to power supply lines and/or communication lines via the adapter element.
